(19) United States
(12) Patent Application Publication
(10) Pub. No.: US 2010/0317731 A1
(43) Pub. Date: Dec. 16, 2010

(54) Hyperhidrosis treatment

(76) Inventor: Elias K. Shaya, Lutherville, MD (US)

Correspondence Address: Sam Rosen, Esquire;HODES, PESSIN & KATZ, P.A., Suite 400, 901 Dulaney Valley Road, Towson, MD, 21204, US

(21) Appl. No.: 12/802,652

(22) Filed: Jun. 11, 2010

Related U.S. Application Data
(60) Provisional application No. 61/186,535, filed on Jun. 12, 2009.

(51) Int. Cl. A61K 31/35; A61P 25/08

(52) U.S. Cl. 514/454

(57) ABSTRACT

Disclosed is a medicinal composition and method of use conspiring topiramate for treating drug-induced excessive sweating (hyperhidrosis). As a further embodiment of this invention, topiramate may be administered along with another medicament that causes excessive sweating to prevent such excessive sweating. As still a further embodiment of this invention, topiramate may be administered along with another medicament that treats excessive sweating, such that the synergy of the drugs effectively treats the sweating.

### RELATED APPLICATIONS

This application is related to provisional patent application Ser. No. 61/186,535 filed Jun. 12, 2009, and is also related to application attorney docket 027969.002 titled TREATMENT FOR MENOPAUSAL AND PERIMENOPAUSAL VASOMOTOR SYMPTOMS, filed concurrently this day.

### FIELD OF THE INVENTION

The herein disclosed invention finds applicability in the field of medicine and for treating excessive sweating (hyperhidrosis) and particularly for treating drug induced sweating.

The present disclosure relates to improvements in the treatment of hyperhidrosis. Most particularly, the invention relates to delivery of a pharmaceutically acceptable amount of an anticonvulsant drug, or prodrug or metabolite thereof.

### BACKGROUND OF THE INVENTION

Hyperhidrosis, or excessive sweating, is a condition experienced by many patients. It can be idiopathic (i.e., primary hyperhidrosis), associated with various other conditions, or caused as a side effect of treatments directed at other patient ailments. For example, excessive sweating has also been identified as a common side effect caused by treatment with antidepressants, choline esterase inhibitors (e.g., Aricept, Exelon) and similar medications (duloxetine, bupropion, escitaloprom, and others).

### OBJECTS OF THE INVENTION

An object of this invention is to produce a therapy which is safe and effective for treating drug-induced hyperhidrosis.

A further object of this invention is to produce a therapy which will mitigate hyperhidrosis caused by a medication that a patient may be taking.

A still further object of this invention is to provide a pharmaceutical composition containing a medical ingredient which causes sweating along with a medicinal agent which will mitigate such sweating.

These and other object of the present invention will become apparent from a reading of the following specification taken in conjunction with the claims.

### SUMMARY OF THE INVENTION

Topiramate (commonly marketed under the brand name Topamax® for the treatment of epilepsy and prevention/treatment of migraine headaches) has been found to have the following, previously undiscovered beneficial pharmacological effects for patients with excessive sweating.

Based on data and observations that the inventor has, he has found topiramate (Topamax®) to have the following, previously undiscovered, beneficial pharmacological effects for patients with excessive sweating:

1. Substantial improvement in excessive sweating which is induced idiopathic or drug-induced sweating.

2. Substantial improvement in excessive sweating caused by treatment with antidepressants and similar medications that are associated with hyperhidrosis.

3. Substantial improvement in excessive sweating that is caused by primary or idiopathic hyperhidrosis.

4. Substantial improvement in excessive sweating caused by other miscellaneous causes of hyperhidrosis.

5. Topiramate is also effective if used on an as needed basis.

The patient population in which the inventor has seen these benefits included both men and women ranging in age from 25 to 79 years. They all benefited with minimal side effects. Their quality of life was substantially improved as they no longer had to change their night clothes or bedding in the middle of the night and they were no longer embarrassed and self conscious at work and at meetings where they would otherwise be sweating profusely while others in the same environment appeared to be quite comfortable.

Excessive sweating can be uncomfortable and embarrassing to patients. Thus, a suitable treatment for patients experiencing excessive sweating is desirable.

The herein disclosed invention describes a method of treating drug-induced hyperhidrosis and is also directed to the use of a combination of drugs which treat excessive sweating.

### DESCRIPTION OF THE INVENTION

The invention is directed to the use of topiramate to treat drug-induced hyperhidrosis (sweating).

Currently, topiramate is marketed in 25 mg tablets, and is indicated for the treatment of epilepsy and migraine headaches. Such patients are directed to take multiple tablets daily, with total daily dosages ranging from about 100-400 mg a day.

According to the present invention, topiramate doses as low as 12.5 mg per day in an adult have been found to be highly effective to treat drug-induced hyperhidrosis. Optionally, the topiramate can be taken as a low dose on an as-needed basis to treat these sweating episodes. Furthermore, as topiramate is known to have strong undesirable side effects, so the low dosage found to be effective in embodiments of the present invention are able to provide effective results without significant incidence of those side effects.

In preferred embodiments of the invention, the topiramate could be provided in a relatively low dosage in an extended release formulation, preferably less than 100 mg daily. Further, for patients diagnosed with night sweats, it may be preferable to direct patients to take topiramate at night.

The inventor became convinced that topiramate was effective in the treatment of excessive sweating. He observed the improvement in patients whose sweating was induced by various antidepressant medications. Some experienced such sweating severity that they had to change their bedding at night as well as their night clothes. Others found it too embarrassing in the day time to the extent that it interfered with their work and social life; and some became reluctant to take their required antidepressant.

The herein disclosed invention involves a method for the treatment of drug-induced excessive sweating comprising administering to a patient experiencing drug-induced excessive sweating an effective amount of topiramate and further wherein the sweating may be an induced idiopathic. The effective amount of topiramate administered is from 12.5 mg to 100 mg. As a feature of this invention, the sweating is a drug induced sweating brought on by antidepressants, or choline esterase inhibitors, such as Aricept, Exelon, duloxetine, bupropion or escitaloprom. As a further feature of this invention the topiramate is administered in an extended release formation. An important feature of this invention is a kit containing mediation comprising as a first component a medicament which when used or its medicinal purpose induces sweating along with topiramate, as second component, to mitigate the sweating affect of the first component. Further the invention can be in the form of a composition in unit-dosage form comprising an effective amount topiramate and a sweat inducing medicament.

As another feature of this invention, topiramate, in effective amounts ranging from 12.5 mg to 100 mg, may be combined with other drugs known to reduce sweating, whereby the patient may realize a reduction in sweating due to the combination of two or more drugs. Drugs with which topiramate may be combined in this manner include boric acid 2-5% tannic acid solutions, resorcinol, potassium permanganate, formaldehyde, glutaraldehyde, methenamine, aluminum chloride (applied topically), botulinum toxin type A, anticholinergic drugs (including oxybutyin, glycopyrrolate, propantheline bromide, and benzatropine), and gabapentin. The topiramate can be administered in an extended release formulation, by mouth, as a skin patch, ointment, or nasal spray. The method of this invention can be practiced using a kit containing medication comprising as a first component a medicament which is useful for treating excessive sweating and as a second component topiramate to impede sweating synergistically, or can be used as a composition in unit dosage form comprising an effective amount of topiramate and a medicament for treating excessive sweating wherein the composition ameliorates the excessive sweating.

### Clinical Studies

In an open label study of 26 patients (18 females and 8 males) who complained of excessive sweating were treated with topiramate on the basis of off label prescribing. The patients were informed of the off-label nature of the treatment and they were counseled about the potential side effects of topiramate. The total daily dose ranged from 25 mg to 100 mg. Over 95% of patients experienced a significant response based on their subjective assessment and report. Patients were all adults with an age range from 25 yrs to 79 yrs. All the patients were on one or more antidepressant including various SSRI's with or without Bupropion. However, several of the patients had experienced excessive sweating for other reasons and before the use of antidepressants, even though adding the antidepressant exacerbated their underlying hyperhidrosis. Some of the underlying causes included perimenopause/menopause as well as primary or idiopathic hyperhidrosis. some had identified vulnerability from before, only made worse by antidepressants.

### CLINICAL EXAMPLES

### Example 1

Mrs. A. had been diagnosed with the following conditions:
Major Depressive Disorder
Dysthymic Disorder
Premenstrual Headaches
Essential Tremor

In August 2007, she started experiencing worsening in her depressive symptoms which prompted an increase in her Cymbalta dose from 60 mg to 80 mg daily. However, she started experiencing the adverse effect of excessive sweating that was particularly severe at night and that woke her up requiring her to change her night clothes. She had been already taking topiramate, 50 mg daily, for treatment and prevention of frequent headaches, especially premenstrually. She was then advised to increase topiramate to 50 mg twice daily starting on Oct. 12, 2007. On her next visit of Jan. 9, 2008, she reported that after initial improvement with the topiramate increase, the excessive sweating has recurred. Accordingly, she was advised to decrease her Cymbalta back to 60 mg daily with the recognition that adverse effects such as excessive sweating are at times dose dependent. She was then taken off topiramate by another doctor and started on Zonisamide for treatment of her headaches. However, she started experiencing excessive sweating even on the lower daily dose of 60 mg of Cymbalta. She then asked for a retrial of topiramate and was started on 25 mg twice daily. On her last visit of Apr. 21, 2009, she reported that the sweating was much better.

### Example 2

Ms. G. had been diagnosed with the following conditions:
1. Recurrent Major Depressive Disorder
2. Possible Bipolar Disorder
3. Intermittent Explosive Disorder
4. Panic Disorder
5. Peptic Ulcer Disease
6. Restless Leg Syndrome
7. Chronic Leg and Back Pain
   She was 54 years old when she presented with the complaint of excessive sweating in the context of possible perimenopausal state and further exacerbated by some of the following medications:
1. Cymbalta 60 mg/d
2. Wellbutrin XL 300 mg/d
3. Xanax 0.5 mg twice daily
4. Lyrica 75 mg twice daily
5. Requip 0.5 mg at bedtime
6. Ambien 10 mg at bedtime

Of these medications, Cymbalta and Wellbutrin are well known for causing the side effect of excessive sweating.

After proper counseling and informed consent, Mrs. G. took a trial of topiramate 25 mg daily, starting on Oct. 1, 2007. On her next visit of Dec. 4, 2007, she reported that the sweating was “much better”. On her last visit of May 29, 2009 she was still taking the topiramate 25 mg per day with maintained benefits that she remained satisfied with and continues to report no adverse effects.

### Example 3

Mr. P. was 27 years old when he presented with the complaint of excessive sweating due to the increase in his Effexor XR dose to 300 mg daily, for more effective treatment of a Major Depressive Disorder exacerbation episode. He was also on Methadone 60 mg daily for treatment of chronic back pain and Lunesta for insomnia. He was started on topiramate 50 mg daily and on follow up visits; he reported “much improvement” in his sweating and no adverse effects from the topiramate.

### Example 4

Mrs. H. had been stable on Zoloft (sertraline) for treatment of Panic and Major Depressive Disorders with a satisfactory remission for several years. However, she was 55 years old when she presented with the complaint of excessive sweating, in October 2007. She wanted to consider switching from Zoloft to a substitute due to the intolerably excessive perspiration. Due to the stability of her conditions and the good response to Zoloft, as well as the possibility that menopausal factors may have been exacerbating her excessive sweating, she decided after counseling and informed consent to take a trial of topiramate 25 mg twice daily. On follow up, she confirmed that the excessive sweating was improved with topiramate and that she has found it to be effective when taken on an as needed basis, depending on her expectations of physical activity and weather/temperature conditions. She reported no adverse effects with the use of topiramate.

### Example 5

Mr. T. is a 56 year old African American male who had a rather complex course of multiple conditions including:
1. Major Depressive Disorder with possible psychotic features.
2. Obesity
3. Sleep Apnea
4. Hypertension
5. Testosterone deficiency

He has also experienced excessive sweating for many years that has been made worse by several of the medications that he takes and the increase in his weight. His medications included:
1. Wellbutrin XL
2. Cymbalta
3. Pristiq
4. Lunesta
5. Provigil
6. Bystolic
7. Testosterone patch
8. Deplin

After counseling and with informed consent, he took a trial of topiramate starting at a daily dose of 50 mg with suboptimal improvement in his excessive sweating. However, the response became satisfactory for him when he increased the topiramate dose to 50 mg twice daily and he has continued to report improvement for five months thereafter.

### Example 6

Mr. B. is a 32 y.o. Caucasian male who has been diagnosed with Major Depressive Disorder and that has been in full remission on treatment with Wellbutrin XL and Cymbalta. However, he has experienced the adverse effect of excessive sweating which has in interfered severely with his job as a salesman—a pharmaceutical representative. He reports feeling self conscious and embarrassed at meetings. After counseling and informed consent, he was started on topiramate 25 mg daily and has continued to take it with satisfactory resolution of his excessive perspiration and without any adverse effects, for over eighteen months.

### Example 7

Mr. M. was 81 y.o. with the following diagnoses, amongst others:
1. Alzheimer's Dementia
2. Major Depressive Disorder
3. Spinal Stenosis
4. Coronary Artery Disease
5. On Cardiac Pacemaker
6. Mild renal Insufficiency
7. Peripheral Neuropathic Pain
8. Sleep Apnea

He was on the following medications:
1. Aricept
2. Cymbalta
3. Plavix
4. Ecotrin
5. Diovan
6. Lanoxin
7. Lasix
8. Allopurinol
9. Singular
10. Gabapentin
11. Foridil
12. Asthmanex
13. Mucinex
14. CoEnzyme Q-10

He presented with excessive sweating disrupting his sleep and causing him to change his clothes at night. This was probably caused by some of his medications including Aricept and Cymbalta amongst others, even though his daily Cymbalta dose was only 30 mg. attempts to decrease his Cymbalta dose caused a recurrence and worsening of his depressive and back pain symptoms. Consequently and after counseling and informed consent, with the patient and his wife, he was started on topiramate 25 mg at bedtime and later increased to 50 mg at bedtime with complete resolution of his excessive sweating and no adverse effects complaints and he stated “it is great”.

Obviously, many modifications may be made without departing from the basic spirit of the present invention. Accordingly, it will be appreciated by those skilled in the art that within the scope of the appended claims, the invention may be practiced other than has been specifically described herein.

What is claimed is:

1. A method for the treatment of excessive sweating wherein the sweating is induced idiopathic or a drug-induced sweating comprising administering to a patient experiencing such excessive sweating an effective amount of topiramate.

2. The method of claim 1, wherein the effective amount of topiramate administered is from 12.5 mg to 100 mg.

3. The method of claim 1, wherein the sweating is a drug induced sweating brought on by antidepressants, choline esterase inhibitors.

4. The method of claim 3, wherein the sweating is a drug induced sweating brought about Aricept, Exelon, duloxetine, bupropion or escitaloprom.

5. The method of claim 2, wherein the topiramate is administered in an extended release formulation.

6. A kit containing medication comprising as a first component a medicament which when used for its medicinal purpose induces sweating along with topiramate, as second component, to mitigate the sweating affect of the first component.

7. A composition in unit-dosage form comprising an effective amount topiramate and a sweat inducing medicament.

8. A kit containing medication comprising as a first component a medicament which treats excessive sweating, along with topiramate, as a second component, to synergistically treat the excessive sweating.

9. A composition in unit-dosage form comprising an effective amount of topiramate and a medicament which treats excessive sweating.
